(19) United States
(12) Patent Application Publication
(10) Pub. No.: US 2018/0087369 A1
(43) Pub. Date: Mar. 29, 2018

(54) Degradable Devices With Assured Identification of Removal

(71) Applicant: Terves Inc., Euclid, OH (US)

(72) Inventors: Andrew Sherman, Mentor, OH (US); Brian Doud, Cleveland Heights, OH (US)

(21) Appl. No.: 15/701,701

(22) Filed: Sep. 12, 2017

Related U.S. Application Data
(60) Provisional application No. 62/398,867, filed on Sep. 23, 2016.

(51) Int. Cl. E21B 47/00; G01N 33/24; E21B 29/00

(52) U.S. Cl. CPC E21B 47/00; E21B 33/12; E21B 29/00; G01N 33/24

(57) ABSTRACT

The use of degradable or dissolvable tools has become a more common practice in subterranean operations for such applications as temporarily isolating zones or diverting flow. A major concern of operators in using degradable tools is the ability to ensure that the tool has completely degraded and is no longer blocking or obstructing flow. This issue can be resolved through the use of degradable or dissolving tools and devices that include one or more tracer elements (e.g., tracer chemicals, chemical elements, particles, tags [RFID, physical tag, microdevice, etc.], etc.) that are released upon the partial or full dissolution of the degradable component, and which can be detected at the surface to ensure the desired degradation or removal of the degradable component as well as hydraulic access to that stage.

The present application claims priority on U.S. Application Ser. No. 62/398,867 filed Sep. 23, 2016, which is incorporated herein by reference.

The present invention relates to the enhanced use of degradable or dissolving tools and devices used in subterranean operations such as drilling, completion, and stimulation operations used in enhanced geothermal, oil and gas, and waste disposal (injection) operations. In particular, the invention relates to degradable components that include one or more tracer elements (e.g., tracer chemicals, chemical elements, particles, tags [RFID, microdevice, etc,] etc.) that are released upon the partial or full dissolution of the degradable component, and which can be detected at the surface to ensure the desired degradation or removal of the degradable component.

### BACKGROUND OF THE INVENTION

Dissolvable and degradable materials have been developed over the last twenty (20) years for the purpose of making well completion and stimulation operations more effective and efficient. Initially, soluble salts were used for temporarily diverting flows and to control tool actuation. This technology was followed by the development and introduction of dissolvable polymers which provided structural performance, thereby enabling applications in such tools as frac balls to operate shifting tools and isolate zones. More recently, dissolvable metals, including high strength magnesium and aluminum alloys, have been developed to enable the production of complete packer and plug fabrication. Pumpable versions of these dissolvable metals have been developed (e.g., flakes, fibers, and beads) for diversion within the fractures outside the liner or wellbore.

Increasingly, a large number of stages are used in completing a well, and longer and higher deviation laterals are produced using directional drilling. These long laterals, deep wells, and high deviations increase costs and difficulties for intervention activities (such as drill-out or retrieval of plugs) and often exceed the distances where coiled tubing intervention services can be effectively used. Components made of degradable and/or dissolvable materials are increasingly being accepted in these applications.

One of the difficulties in using components formed of degradable and/or dissolvable materials is that sometimes such components have been known to not degrade or not properly degrade. Most of these components formed of degradable and/or dissolvable materials require the presence of brine to cause the degrading and/or dissolving of the component. If gas pockets, tar, or other contaminants block access to the components formed of degradable and/or dissolvable materials, or if the salt content, temperature, or conditions of the brine are wrong for the proper degrading and/or dissolving of the component, the component can remain in the well and flow communication in the well can be reduced or prevented.

Components formed of degradable and/or dissolvable materials can normally be easily drilled out or otherwise removed (if accessible). Oftentimes, the operator will not be aware of a problem associated with a component that has not degraded or dissolved or has not properly degraded or dissolved, particularly if the problem occurs at a toe stage in a well with a large number of stages or zones.

Although the use of tools formed of degradable and/or dissolvable materials has become a more common practice in subterranean operations for such applications as temporarily isolating zones or diverting flow, a major concern of operators in using such tools is the ability to ensure that such tool has completely degraded or dissolved and is no longer fully or partially blocking flow in a well. As such, it is therefore highly desirable for a method in which the operator conclusively knows that the tool formed of degradable and/or dissolvable materials has been properly removed from a well.

In view of the current state of the art, there is a need for the use of tracer chemicals, elements, or tags (RFID) in components that are released upon dissolution of the degradable component and which can be detected at the surface to ensure tool removal.

### SUMMARY OF THE INVENTION

The present invention relates to degradable and/or dissolving tools or devices (herein after referred to as a “degradable component”) and the use thereof in subterranean operations such as drilling, completion, and stimulation operations used in geothermal, oil and gas, and waste disposal (injection) operations, wherein the degradable component includes one or more tracer elements (e.g., tracer chemicals, chemical elements, particles, tags [RFID, microdevice, etc.], etc.) that are released upon the partial or full dissolution of the degradable component, and which the one or more tracer elements can be detected at the surface of the proper removal or degradation of the degradable component. Non-limiting examples of the types of tools used in geothermal, oil and gas, and waste disposal (injection) operations that can be formed of or incorporate a degradable component are disclosed in U.S. Pat. Nos. 8,905,147; 8,717,268; 8,663,401; 8,631,876; 8,573,295; 8,528,633; 8,485,265; 8,403,037; 8,413,727; 8,211,331; 7,647,964; US Publication Nos. 2015/0239796; 2015/0299838; 2015/0240337; 2016/0137912; 2013/0199800; 2013/0032357; 2013/0029886; 2007/0181224; and WO 2013/122712; which are all incorporated herein by reference. The use of degradable components has become a more common practice in subterranean operations for such applications as temporarily isolating zones or diverting flow. A major concern of operators in using degradable components regarding the ability to ensure that the degradable component has sufficiently or completely degraded can be resolved through the addition of tracer elements that are released upon dissolution of the degradable component and which can be detected to ensure that the degradable component has been sufficiently removed. Tracer elements can be released as ions/atoms, molecular or particles species, or can be discreet devices such as RFID microchips, etc. The one or more tracer elements can be incorporated uniformly throughout the degradable component, added to specific locations in the degradable component, or placed at different depths within the degradable component. A degradable component can include a single tracer element or different tracer elements. The tracer element can be uniformly dispersed in the degradable component or in one or more regions of the degradable component or be concentrated in one or more regions of the degradable component.

In one non-limiting aspect of the present invention, a degradable component includes the addition of one or more tracer elements in an interior of the degradable component for the purpose of verifying and/or assuring that the degradable component has sufficiently degraded and/or dissolved. The tracer element is generally no more than 12700 microns in size. In one non-limiting embodiment, the tracer element in the form of a magnetic particle, nanowire, nanocomposites, nanohorns, functionalized nanotubes, metalized nanotubes, magnetic wires, piezoelectric materials, fluorescing particle, phosphorescent compound and/or particles, compounds or molecules that can include stable isotopes, radioactive isotopes, rare earth or other specific elements generally have an average size of less than about 10 microns in size, typically 0.001 less than 10 microns (and all values and ranges therebetween), more typically less than 5 microns, still more typically less than one micron, and yet more typically less than 0.5 micron in size (e.g., nanoparticle [1-100 nm and all values and ranges therebetween]); however, this is not required. In another non-limiting embodiment, tracer elements in the form of microRFID, micro-resonant device (MRD) can have a size that is generally less than about 10000 microns and typically about 0.01 to 8000 microns (and all values and ranges therebetween). The type and/or amount of one or more tracer elements used in a particular component is non-limiting. A component can include the same or have different types of tracer elements in the particular component. The tracer elements can be 1) uniformly dispersed throughout a particular component, 2) concentrated in one or more regions of a particular component, and/or 3) include different types of tracer elements in different regions of a particular component. In one one-limiting embodiment, the tracer element is incorporated in the degradable component and is designed to be released during or after the partial or full degradation of the degradable component. In another and/or non-limiting embodiment, one or more tracer elements are placed in an internal cavity of the degradable component and a degradable or non-degradable plug or cap is used to close the cavity. The plug or cap can have the same or different composition from the degradable component. In another and/or alternative non-limiting embodiment, the degradable component is configured to release a concentrated amount of tracer elements over a short period after the degradable plug or cap has partially or fully dissolved or degraded. The one or more cavities in the tool can be formed by machining. The one or more cavities can be closed by use of a plug, wherein the plug is connected to the cavity by a threaded connection, interference fit, swaged connection, etc. The tracer element can be designed, after the degradable component partially or fully degrades, to release from the degradable component and be carried with fluid flow to a location at some distance from where such one or more tracer elements are released from the degradable component, and which tracer elements can be detected once such tracer elements are transported to a different location from the location of the degradable component. In another and/or alternative embodiment, different tracer elements can be used in different regions or zones of a degradable component to provide information as to the degree to which a degradable component has degraded and/or whether a particular region of a degradable component has degraded and/or the degree to which it has been degraded. For example, a degradable component can include a certain amount of tracer element. By measuring the amount of tracer element that has been detected at the surface, an estimation or calculation can be made regarding the degree to which the degradable component has degraded and/or the degree to which multiple degradable components have degraded. In another example, different types of tracer element are incorporated and/or positioned at different regions of a degradable component. By measuring and/or detecting the tracer element that has been detected at the surface, it can be determined whether a certain region of one or more degradable components have begun to degrade and to what degree that a certain region of one or more degradable components have degraded. In another and/or alternative embodiment, different tracer elements can be used in different degradable components. As such, when multiple degradable components are positioned in a well, etc., the measuring and/or detecting of a particular tracer element and/or volume of tracer element at the surface can be used to determine whether 1) a particular degradable component(s) has begun to degrade or has degraded, 2) whether a certain region of a particular degradable component(s) has begun to degrade, and/or 3) to what degree that the particular degradable component(s) or a certain region of the particular degradable component(s) has degraded.

In another and/or alternative aspect of the present invention, the tracer element can be chosen from one or more tracer elements which can include microRFID, magnetic wires, nanowires, magnetic particles, fluorescing, and phosphorescent compounds and/or particles; and/or from compounds or molecules that can include stable isotopes, radioactive isotopes, rare earth or other specific elements, as well as compounds with high sensitivity in mass spectroscopy or other analytical technique that is sensitive to ppb levels. A variety of detectable materials can be used as the tracer element such as trackers, taggants, markers, tracking materials, and/or tracers.

In another and/or alternative aspect of the present invention, the tracer element can be a material as disclosed in U.S. Pat. No. 8,006,755 (e.g., piezoelectric materials with a perovskite crystallographic structure type such as lead zirconate titanate (PZT) and barium titanate; magnetostrictive materials such as Terfenol-D (a family of alloys of terbium, iron and dysprosium), Samfenol (a family of alloys of samarium and iron, sometimes also containing other elements such as dysprosium), and Galfenol (a family of alloys of gallium and iron, sometimes also containing other elements); U.S. Pat. No. 7,516,788 (e.g., a dye detectable by color such as “acid blue” water-soluble dyes, “oil red” oil-soluble dyes, molecular iodine, iron oxide class pigments, chrome oxide pigments, mica ferric oxide pigments, other oxide or inorganic pigments, or organic pigments; marker easily detected spectrographically such amides, amines, or phenols); U.S. Pat. No. 6,725,926 (e.g., water soluble salts such as metal salts in which the metal is selected from Groups Ito VIII of the Periodic Table of the Elements as well as the lanthanide series of rare earth metals, barium, beryllium, cadmium, chromium, cesium, sodium, potassium, manganese, zinc, barium bromide, barium iodide, beryllium fluoride, beryllium bromide, beryllium chloride, cadmium bromide, cadmium chloride, cadmium iodide, cadmium nitrate, chromium bromide, chromium chloride, chromium iodide, cesium bromide, cesium chloride, sodium bromide, sodium iodide, sodium nitrate, sodium nitrite, potassium iodide, potassium nitrate, manganese bromide, manganese chloride, zinc bromide, zinc chloride, zinc iodide, sodium monofluoroacetate, sodium trifluoroacetate, sodium 3-fluoropropionate, potassium monofluoroacetate, potassium trifluoroacetate, potassium 3-fluoropropionate); U.S. Pat. No. 7,921,910 (e.g., the lanthanide series of rare earth metals, strontium, barium, gallium, germanium, and combinations thereof, particularly, lanthanum, cerium, strontium, barium, gallium, germanium, tantalium, zirconium, vanadium, chromium, manganese, and combinations thereof, especially lanthanum, cerium, and combinations thereof, ZrSiO4, ZnO, SrO(CO₂), Nd₂O₅, Pr₆O₁₁, MnO, CuO, Cr₂O₃, NiO, V₂O₅, Co₃O₄, Sb₂O₃, La₂O₃, CeO₂); and U.S. Pat. No. 6,991,780 (e.g., aluminum-zirconium antiperspirant salt compositions such as Zr(OH)_{4-b}X_{b} wherein X is Cl, Br, I, or NO₃; and b is about 0.7 to about 4.0), all of which are incorporated herein). Methods and apparatuses for detecting the tracer elements in accordance with the present invention include the systems, devices, methods, and apparatuses such as inductively-coupled plasma (ICP), X-ray fluorescence, or proton-induced X-ray emission (PIXE), chemical analysis, etc.

In another and/or alternative aspect of the present invention, the tracer element can optionally be in the form of one or more nanomaterials and/or types of nanomaterials such as, but not limited to, nanotubes, nanocomposites, nanohorns, functionalized nanotubes, metalized nanotubes, combinations of different nanomaterials, and combinations of different functionalized nanotubes and/or metalized nanotubes, e.g., functionalized nanotubes as disclosed in U.S. Pat. No. 7,858,691 (e.g., carbon nanotubes surface functionalized with oxygen-bearing molecules); U.S. Pat. No. 7,854,945 (e.g., functionalized carbon nanotubes); U.S. Pat. No. 8,062,702 (e.g., coated fullerene comprising a layer of at least one inorganic material covering at least a portion of at least one surface of a fullerene; and at least one composite matrix selected from the group consisting of polymers, ceramics and inorganic oxides); U.S. Pat. No. 7,968,489 (Carbon nanotubes, also known as fibrils, are vermicular carbon deposits having diameters less than 1.0 μ, generally less than 0.5 μ, and typically less than 0.2 μ. Carbon nanotubes can be either multi walled [i.e., have more than one graphene layer more or less parallel the nanotube axis] or single walled [i.e., have only a single graphene layer parallel to the nanotube axis]. Other types of carbon nanotubes are also known, such as fishbone fibrils [e.g., wherein the grapheme layers exhibit a herringbone pattern with respect to the tube axis], etc. Carbon nanotubes may be in the form of discrete nanotubes, aggregates of nanotubes [i.e., dense, microscopic particulate structure comprising entangled carbon nanotubes] or a mixture of both. Carbon nanotubes are distinguishable from commercially available continuous carbon fibers. Carbon fibers have aspect ratios (L/D) of at least 10⁴and often 10⁶ or more, while carbon nanotubes have desirably large, but unavoidably finite, aspect ratios [e.g., less than or greater than 100]. The diameter of continuous carbon fibers, which is always greater than 1.0 μ, and typically 5 to 7 μ, is also far larger than that of carbon nanotubes, which is usually less than 1.0 μ. Carbon nanotubes also have vastly superior strength and conductivity than carbon fibers.); U.S. Pat. No. 6,905,667 (carbon nanotube surfaces are functionalized in a non-wrapping fashion by functional conjugated polymers that include functional groups. The polymers can be noncovalently bonded with carbon nanotubes in a non-wrapping fashion. The polymers can be provided having a relatively rigid backbone that is suitable for noncovalently bonding with a carbon nanotube substantially along the nanotube's length, as opposed to about its diameter. Examples of rigid functional conjugated polymers that may be utilized in embodiments of the present invention include, without limitation, poly(aryleneethynylene)s and poly(3-decylthiophene). The polymers can comprise at least one functional extension from the backbone for functionalizing the nanotube.); U.S. Pat. No. 7,771,696 (A composition is provided in which carbon nanofibers are functionalized with at least one moiety where the moiety or moieties comprise at least one bivalent radical. The composition can include a nanocomposite, such as polyimide films. Carbon nanofiber (CNF) includes all varieties of carbon nanofibers, including all types of internal and external structures. Examples of internal structures include, but are not limited to, arrangement of the graphene layers as concentric cylinders, stacked coins, segmented structures, and nested truncated cones. Examples of external structure include, but are not limited to, kinked and branched structures, amount and extent of surface rugosity, diameter variation, nanohorns, and nanocones. CNFs also include structures that have a hollow interior and those that do not. The hollow core, if it exists, can have a diameter of 20 and above, or 20-490 nm, or 30-190 nm, or 50-190 nm, or 50-90 nm. CNFs can have an outer diameter dimension of 30 nm and above, or 30-500 nm, or 40-200 nm, or 60-200 nm, or 60-100 nm. Aspect ratios for CNFs can be 500 and above, or 800 and above, or 1000 and above.); U.S. Pat. No. 7,459,137 (Functionalizing carbon nanotubes by reacting them with organic functionalizing agents in the absence of solvent [“solvent-free” conditions]. Carbon nanotubes can comprise both multi- and single-wall varieties. They can be produced by any known technique and can be of any length, diameter, or chirality which suitably provides for carbon nanotubes functionalized under solvent-free conditions. Samples of carbon nanotubes can comprise a range of lengths, diameters, and chiralities, or the nanotubes within the sample may be largely uniform. The samples may also be in the form of “ropes” or macoscopic mats called “bucky paper. Functionalization comprises attaching organic and/or organometallic moieties to the carbon nanotubes at their ends, their sidewalls, or both. Generally, this functionalization involves a covalent bond between the functional moiety and the carbon nanotube and it is accomplished by reacting the carbon nanotubes with an organic functionalizing agent. An organic functionalizing agent may be any species that suitably functionalizes carbon nanotubes under solvent-free conditions. Organic functionalizing agents include, but are not limited to, diazonium species; aryl radicals; alkyl radicals; aryl carbocations; aryl carbanions; alkyl carbanions; alkyl carbocations; 1,3-dipoles; carbenes; heteroatom-containing radicals, cations, and anions; ylides; benzyne; dienes; dienophiles, and combinations thereof. Organic fuctionalizing agents may further include organometallic species such as organozincates, carbenes, Grignard reagents, Gillman reagents, organolithium reagents, and combinations thereof.); U.S. Pat. No. 7,241,496 (carbon nanotube surfaces are functionalized in a non-wrapping fashion by functional conjugated polymers that include functional groups. Polymers that are noncovalently bonded with carbon nanotubes in a non-wrapping fashion can be used. Polymers can be provided that comprise a relatively rigid backbone that is suitable for noncovalently bonding with a carbon nanotube substantially along the nanotube's length, as opposed to about its diameter. The major interaction between the polymer backbone and the nanotube surface can be parallel π-stacking. The polymers can comprise at least one functional extension from the backbone that are any of various desired functional groups for functionalizing a carbon nanotube. Carbon nanotubes are elongated tubular bodies which are typically only a few atoms in circumference. The carbon nanotubes are hollow and have a linear fullerene structure. The length of the carbon nanotubes potentially may be millions of times greater than their molecular-sized diameter. Both single-walled carbon nanotubes (SWNTs), as well as multi-walled carbon nanotubes (MWNTs) can be used); U.S. Pat. No. 6,203,814 (Graphitic nanotubes, which includes tubular fullerenes (commonly called “buckytubes”) and fibrils, are functionalized by chemical substitution or by adsorption of functional moieties. The graphitic nanotubes which are uniformly or non-uniformly substituted with chemical moieties or upon which certain cyclic compounds are adsorbed and to complex structures comprised of such functionalized fibrils linked to one another.); U.S. Pat. No. 8,058,364 (Free-radical addition reactions which graft [i.e., chemically bond] molecules onto the nanoscale fibers' surfaces with minimal effect on the mechanical properties of the nanoscale fibers themselves. “Chemically bonded” or “chemical bond” refers to covalent bonds or ionic bonds between molecules and the atoms on the nanoscale fibers' surfaces resulting from a chemical reaction of the molecules and the atoms on the nanoscale fibers' surfaces. Examples of chemical bonds include covalent bonds and ionic bonds such as negatively charged SWNT/Li⁺ bonding.); and U.S. Pat. No. 7,976,816 (Functionalizing the wall of single-wall or multi-wall carbon nanotubes by use of acyl peroxides to generate carbon-centered free radicals to allow for the chemical attachment of a variety of functional groups to the wall or end cap of carbon nanotubes through covalent carbon bonds without destroying the wall or endcap structure of the nanotube. Carbon-centered radicals generated from acyl peroxides can have terminal functional groups that provide sites for further reaction with other compounds. Organic groups with terminal carboxylic acid functionality can be converted to an acyl chloride and further reacted with an amine to form an amide or with a diamine to form an amide with terminal amine. The reactive functional groups attached to the nanotubes provide improved solvent dispersibility and provide reaction sites for monomers for incorporation in polymer structures. The nanotubes can also be functionalized by generating free radicals from organic sulfoxides. Sidewall functionalizing of single-wall carbon nanotube comprises decomposing a diacyl peroxide in the presence of carbon nanotubes wherein the decomposition generates carbon-centered free radicals that react and form covalent bonds with carbon in the single-wall carbon nanotube wall to form a single-wall carbon nanotube sidewall functionalized with at least one organic group through a carbon bond to the nanotube. An acyl peroxide, also known as a diacyl peroxide, is a compound with a structure of the type RC(O)OOC(O)R′, where R and R′ groups can be either alkyl or aryl. The acyl peroxide can be an aroyl peroxide wherein the R or R′ group comprises an aromatic component. The acyl peroxide can be an aroyl peroxide and comprises benzoyl peroxide, which, upon decomposition, liberates carbon dioxide and generates phenyl radicals that attach to the sidewalls of the nanotubes to form sidewall phenylated single-wall carbon nanotubes.), all of which are incorporated herein.

In another and/or alternative aspect of the present invention, the tracer element can be an identifier tag that includes one or more RFID, micro-resonant device (MRD) and/or other tag-device. A variety of RFID devices are disclosed in US Publication No. 2010/0007469 (A nano RFID device or tag may be less than about 150 nanometers in size. The nano RFID device may be a passive, active or semi-passive nano RFID device. The nano RFID device may include a nano antenna that may comprise one or more carbon tubes. The nano RFID device may include a nano battery. The nano RFID device may include an environmentally reactive layer that reacts to its immediate environment to affix or adhere to a target. Most common RFID tags typically contain at least two parts. One is an integrated circuit for storing and processing information, modulating and demodulating a radio frequency (RF) signal, and other specialized functions. The second part is an antenna for receiving and transmitting a signal. A technology called “chipless RFID” allows for discrete identification of tags without an integrated circuit, thereby allowing tags to be printed directly onto assets at a lower cost than traditional tags. Passive RFID tags typically have no internal power supply. The electrical current induced in the antenna by the incoming radio frequency signal provides just enough power for the CMOS integrated circuit in the tag to power up and transmit a response. Most passive tags signal by backscattering a carrier wave from a reader. This may mean that the antenna has to be designed both to collect power from the incoming signal and also to transmit the outbound backscatter signal. The response of a passive RFID tag is not necessarily just an ID number; the tag chip can contain non-volatile, perhaps writable, EEPROM for storing data. Semi-passive tags are similar to active tags in that they have a power source, but it may only power the micro-circuitry and may not power the broadcasting of the signal. The response may be powered by the backscattering of the RF energy from the reader.); US Publication No. 2010/0001841 (An RFID device (RFID tag) of about 150 nanometers or smaller in dimension. The RFID device may include semiconductors as small as is 90-nm, perhaps with some chips configured and provided at the 65-nm, 45-nm and/or 30-nm size level. The technology for the included electrical circuitry may include CMOS or related technology for low power consumption. A nano RFID device constructed by nanotechnology techniques provides advantages over the currently available RFID devices such as permitting the RFID device to be distributed by airborne, ingestion, or contact distribution (perhaps by aerosol or a mist, for example), or constructed to react to an specific environmental factor for embedded/affixing to a surface or specific type of material (e.g., an organic material). This provides for dynamic distribution of the RFID device to track targeted subjects or objects.); and US Publication No. 2010/0001846, all of which are incorporated fully herein, can be used as the tracer element. A variety of micro-resonant devices are disclosed in US Publication No. 2009/0027280 (A micro-resonant device (MRD) that generate resonance at radio frequencies. These individual, often monolithic, devices can be located in three-dimensional space and tracked anywhere in a target area using a conventional MRI scanner or other transducers, e.g., radiofrequency transducers. The MRDs generate high-sensitivity contrast in conventional MRI scanners, have a diameter of anywhere from a few nanometers to 1000 microns, and can be manufactured using micro-mlectro-mechanical systems (MEMS) technology. The devices are optionally coated to isolate them from the environment. The monolithic MRDs can include an antenna component that receives an excitation signal and transmits an emission signal; and a resonator component that receives an excitation signal and generates a corresponding emission signal; and, optionally an outer coating that envelopes the device and isolates the device from its environment. These devices have an overall diameter of less than about 1000 microns, e.g., 100 or 10 microns, and a Q value of greater than about 5, e.g., greater than 10, 50, 100, or much higher, and the emission signal is (i) a resonant frequency of the device emitted at a delayed time compared to the excitation signal (or at a time after the excitation signal has stopped), (ii) a frequency different than the excitation signal; (iii) a signal at a different polarization than the excitation signal, or (iv) a resonant frequency of the device (when the device is tuned to the same frequency as the nuclei being imaged) which upon excitation by an excitation field (e.g., a magnetic field), distorts the applied excitation field. The antenna component and the resonator component can be the same component, i.e., one component that functions as both an antenna and as a resonator. When the coating is present, the coating can be cross-linked, and the carbon can be or include amorphous carbon, diamond, or nano-crystalline diamond. The MRDs can be designed such that the resonant frequency is proportional to an applied magnetic field, e.g., by fabricating the resonator of a magnetic metal or alloy to induce magnetic field dependence to the resonant frequency. The MRD can be in the form of cylindrical or prismatic length extender bars that include a transducer material, e.g., a piezoelectric or magnetostrictive transducer material, and that have a length of less than about 100 microns and a diameter of less than about 100 microns; and optionally an outer coating that envelopes the device and isolates the device from its environment. The MRD can resonate at a resonant frequency of greater than about 50 MHz after receiving an excitation signal at the resonant frequency. The resonant frequency can be greater than about 400 MHz, greater than about 2 GHz, or even greater than 1 THz. The MRD can be in the form of devices that include a hermetically-sealed housing having walls forming an internal chamber, a cantilever arranged within the internal chamber and having a free end and a fixed end connected to a wall of the housing, and an electrode arranged within the internal chamber in parallel and spaced from the cantilever; wherein the overall size of the device is no larger than about 1000 microns, e.g., no larger than 100 or 10 microns. The cantilever and the electrode can each be made of silicon (e.g., polysilicon) and the housing can include silicon nitride. The cantilever and electrode can be made of the same material, or different materials, e.g., with different electron work functions. For example, one material of the cantilever or electrode can be silicon doped N and a second material of the electrode or cantilever can be silicon doped P. The cantilever can be made of a magnetic metal or alloy to induce magnetic field dependence to the resonant frequency. The MRD can be in the form of a sandwich of at least two layers rolled into a cylinder, wherein a first layer includes a conductor and a second layer comprises an insulator; wherein the device has an overall diameter of less than 5 mm and a Q value of greater than 5 and wherein, when exposed to an excitation signal at a resonant frequency of the device, the device generates an emission signal comprising the resonant frequency for a time after the excitation signal has ended. The MRD can include a third magnetic layer made of, e.g., iron, nickel, cobalt, or alloys thereof, or other magnetic materials described herein. The MRD can include an outer coating that envelopes the device and isolates the device from its environment. The MRD can be in the form of planar L-C resonator devices that include a spiral inductor and a thin-film capacitor. The new MRD can be manufactured in the form of piezoelectric cantilever resonator devices having a loop antenna. The MRD can be tracked by generating an excitation signal in a target area in which the device might be located; receiving an emission signal from the one or more MRDs, if any, in the target area; and processing the emission signal to determine the location of the device. The MRD can be imaged by processing the emission signal and generating an image from the processed emission signal. The MRDs can have an overall diameter of about 10 microns or less. The emission signal can be a resonant frequency of the MRD, and the device can further include a magnetic material to induce magnetic field dependence to the resonant frequency. The emission signal can be a frequency of at least 100 MHz, e.g., 400 MHz, 2 GHz, or 1 or more THz. The MRD can be attached to an object and be used to track the object within a target area. The MRD can include one or more ligands that specifically bind to a target moiety and induce a change in the frequency of the emission signal of the MRD to sense a change in the environment of the target area. The MRD can have an overall outer diameter or dimension of less than about 1000 microns, and can be much smaller, e.g., less than 500, 250, 100, 50, 20, 10, 5, or 1 micron, or even on the nanometer scale, e.g., 500, 250, 200, 100, 50, 25, 10, or 5 nanometers. The MRD can be individual, standalone, monolithic devices, or can be made of a set of nano-resonant devices that are each on the nanoscale, i.e., about 500 nanometers or less, e.g., less than 250, 100, 50, 25, 10, or 5 nanometers in size. The nano-resonant device can either (i) individually produce a resonant signal, and when acting in concert in a particular target location, the set of nano-resonant devices produces a collective signal of sufficient power to be detected in the same way that a signal from a micro-resonant device is detected, or (ii) individually do not produce a signal, but assemble, e.g., self-assemble, at a target location to form a MRD to produce a detectable signal or collectively act like a micro-resonant device to produce a detectable signal. The nano-resonant device can produce a detectable signal and serve as a micro-resonant device, depending on its size and resonant frequency. The MRD can be a passive, robust, solid-state device. The MRD can be designed and fabricated so that its resonant frequency is sensitive to its surrounding temperature, chemistry, pH, or specific target moieties, such as specific ions or chemicals, thus making it useful as local sensors with an RF readout. The MRD can be composed of metallic layers can be detected by conventional computed tomography (CT). The MRDs can act as RF tags to track the MRD.); and US Publication No. 2009/0027280, all of which are incorporated fully herein) can be used as the tracer element. A variety of nano-devices including nano-robots as disclosed in U.S. Pat. No. 8,269,648 (A system for communicating information to nano sensors located within a select subsurface region can be provided wherein a plurality of transmit antennae located at multiple positions on or below the terrain surface, the antennae adapted to transmit immediately in the far field electromagnetic energy beam signals from multiple positions on or below the terrain surface and separated from the select subsurface region via geological strata, the electromagnetic energy beam signals of a predetermined frequency, duration, and power that combine to cover a target area of the select sub surface region; and a plurality of nano sensors located in an oil reservoir at the select subsurface region and responsive to said electromagnetic beam signals to activate a function of the nano sensors. The system can comprise a plurality of receive antennae adapted to receive reflections from the target area in response to the transmitted energy beam signals impinging thereon, wherein the nano sensors are adapted to reflect or absorb the particular frequencies transmitted by the antennae such that the reflections are characteristic of the nano sensors located within the target area being impinged upon by the transmitted far field electromagnetic energy beam signals. Each of the transmit antennae can comprise a compact parametric antenna having a dielectric, magnetically-active, open circuit mass core, ampere windings around said mass core, said mass core being made of magnetically active material having a capacitive electric permittivity from about 2 to about 80, an initial permeability from about 5 to about 10,000 and a particle size from about 2 to about 100 micrometers; and an electromagnetic source for driving said windings to produce an electromagnetic wavefront. A communications method can be provided for communicating information to nano sensors located within a select subsurface region: from multiple positions on or below the terrain surface and separated from the select subsurface region via geological strata, transmitting immediately in the far field electromagnetic energy beam signals of a predetermined frequency, duration, and power that combine to cover a target area of the select sub surface region; and receiving via one or more nano sensors located in an oil reservoir at the select subsurface region said electromagnetic beam signals, wherein the one or more nano sensors are responsive to the received electromagnetic beam signals to activate a function of the nano sensors. The nano sensors can be responsive to the received electromagnetic beam signals to recharge a battery of the nano sensors using the received electromagnetic energy signals. The nano sensors can be responsive to the received electromagnetic beam signals to realign themselves according to the magnetic field impinging thereon. The nano sensors can be responsive to the received electromagnetic beam signals to effect a chemical reaction within the oil reservoir. In another embodiment, the nano sensors are responsive to the received electromagnetic beam signals for initiating communications with other said nano sensors. The nano sensors can be responsive to the received electromagnetic beam signals for retrieving information from memory contained within the nano sensors and transmitting the information. The nano devices can receive the transmitted electromagnetic energy to recharge a power system within the nano devices. The nano devices can be designed to reflect a portion of the energy from the transmissions, wherein the reflected energy related to relative changes in the position of an ensemble of nano devices existing in a given location. A source of electromagnetic energy from an array of antennae transmitting immediately in the far field is provided for imparting pulses, wherein the pulses will be reflected by the nano devices according to the reflectivity to the nano devices material and its location as it may exist. An array of receiver antennae may be used to initially establish a reference of the reflected pattern, and then operated in conjunction with the transmit array to monitor the movement of the nano devices. A source of the electromagnetic energy from an array of antennae transmitting in the far field can be provided for triggering or activating nano devices. A source of electromagnetic energy from an array of antennae transmitting immediately in the far field can be provided for imparting pulses at the depth of the fluid reservoir whereby the returns reflected by nano devices according to the reflectivity to the nano particle or nano sensor material and its location for mapping a 3-dimensional map and over time a 4-dimensional map. A source of electromagnetic energy from an array of antennae transmitting in the far field can be provided for imparting pulses to communicate with nano devices to effect motion of the nano devices.), which is incorporated fully herein, can be used as the tracer element.

In another and/or alternative aspect of the present invention, the tracer element includes one or tracer molecules, and/or one or more different tracer molecules. The one or more tracer molecules can include at least one of fluorescent molecules, UV-active molecules, isotopically enriched molecules (e.g., molecules having mass spectra distinct from non-isotopically enriched molecules, etc.), radiolabeled molecules, radioactive molecules, metal nanoparticles, hydrophobic molecules, hydrophilic molecules, rare earth nanoparticles, phosphorescent or fluorescent nanoparticles, stable isotopes, and combinations thereof. Easily ionizable molecules such as, but not limited to, halogen-containing molecules can also be used as tracer molecules due to their low detection threshold. Flouronated compounds can be used as tracer materials. Sulfonated compounds can be used as tracer materials. Triheptylamine (THA) can be used as a tracer material. THA is a highly hydrophobic molecule due to its long alkyl chains. Furthermore, THA's nitrogen atoms can be easily distinguished by mass spectrometry according to the nitrogen rule, where an odd number of nitrogen atoms will afford an odd mass. The one or more tracer molecules can also include fluorescent dyes, such as 1,5-diphenyloxazole or fluorescein. In one non-limiting embodiment, the one or more releasable tracer molecules can be non-isotopically enriched molecules that are easily detectable by their mass spectra or other unique spectroscopic signature; however, this is not required. In another non-limiting embodiment, the releasable tracer molecules can include metal nanoparticles and molecules that are sensitive to the presence of heavy metals (e.g., chelating ligands, etc.); however, this is not required. In another non-limiting embodiment, the releasable tracer molecules can include molecules that are non-radioactive; however, this is not required. In another non-limiting embodiment, the releasable tracer molecules can include molecules that are radioactive and thus detectable by a scintillation counter; however, this is not required.

In another and/or alternative aspect of the present invention, the tracer element can be incorporated into degradable materials by encapsulation (e.g., polymer, etc.); however, this is not required. The encapsulation of the tracer element (when used) can be used to create controlled release detection and/or allow placement and then release at different depths and within a well formation. For example, one or more tracer elements can be coated with a dissolvable material (e.g., polymer, metal, carbohydrate, sugar, etc.) prior to or after being applied onto or incorporated into a degradable component. The coating is generally formulated to dissolve when exposed to certain environmental conditions (e.g., fluid temperature, fluid composition, etc.).

In another and/or alternative aspect of the present invention, there is provided a system for assuring performance of a degradable component that includes a) a degradable material partially or fully forming the degradable component; and b) one or more tracer elements and/or types of tracer elements incorporated on and/or in the degradable component, wherein the one or more tracer elements are configured to be released from the degradable component when the degradable component partially or fully degrades.

In another and/or alternative aspect of the present invention, there is provided a method of detecting one or more tracer elements and/or types of tracer elements at some distance from a location of a degradable component (e.g., a tool, a component of a tool, a valve, a plug, frac ball, pipe, sleeves,casting, etc.).

In another and/or alternative aspect of the present invention, there is provided a tracer element that is a stable isotope/element that is incorporated on and/or in a degradable component which is detectable using analytical techniques prior to, during, and/or after the degradable component partially or fully degrades.

In another and/or alternative aspect of the present invention, there is provided a tracer element that is an oxide or other type of compound (such as a rare earth oxide) that is incorporated on and/or in a degradable component which is detectable using analytical techniques prior to, during, and/or after the degradable component partially or fully degrades. The average particle size of the compound can be no more than about 10 microns, and typically no more than about 1 micron, and typically no more than about 0.5 micron. The compound can be formulated or be designed to be detectable by various techniques (e.g., detection of highly polar molecules or a radioisotope, an isotope that can be activated, UV-active material, a fluorescent material, die or phosphorescent particles, etc.).

In another and/or alternative aspect of the present invention, there is provided a tracer element that is a rare earth material not normally found in the formation fluids that is incorporated on and/or in a degradable component which is detectable using analytical techniques prior to, during and/or after the degradable component partially or fully degrades.

In another and/or alternative aspect of the present invention, there is provided a tracer element that is incorporated uniformly throughout the degradable component.

In another and/or alternative aspect of the present invention, there is provided a tracer element that is located on and/or within one or more specific areas or regions of the degradable component.

In another and/or alternative aspect of the present invention, there is provided a degradable component having one or more cavities that are formed by machining and then plugged, and wherein the tracer element is positioned in the plugged cavity. The cavity and optional sealing structure for the cavity can be configured to release a portion or all of the tracer element from the cavity after the degradable component has partially or fully degraded (e.g., 5%-100% degradation and all values and ranges therebewteen). Generally, the cavity of the degradable cavity is not designed to allow release of said tracer elements from the cavity until the degradable component has degraded at least about 10%, and typically at least about 20%, and more typically at least about 20%-60%. Generally the size of the cavity is no more than 80 vol. % of the degradable component, and typically about 0.1-80 vol. % (and all values and ranges therebetween) of the degradable component, and more typically about 0.5-60 vol. % of the degradable component, and more typically about 0.75-45 vol. % of the degradable component.

In another and/or alternative aspect of the present invention, there is provided a degradable component having one or more cavities that can be closed by use of a plug, wherein said plug is connected to the cavity by a threaded connection, interference fit, swaged connection, etc. The plug may or may not be formed of a degradable material.

In another and/or alternative aspect of the present invention, there is provided a degradable component having one or more cavities wherein at least one of the cavities includes one or more tracer elements in an amount of at least about 0.01 grams. In one non-limiting embodiment, at least one of the cavities includes one or more tracer elements in an amount of 0.01-10 grams (and all values and ranges therebetween).

In another and/or alternative aspect of the present invention, there is provided a tracer element that is in the form of an RFID tag, magnetic wire, or other information carrying device that is incorporated on and/or in a degradable component which is detectable using analytical techniques prior to, during, and/or after the degradable component partially or fully degrades.

In another and/or alternative aspect of the present invention, there is provided a tracer element that is in the form of a tracer molecule or element that is incorporated on and/or in a degradable component which is detectable using analytical techniques prior to, during, and/or after the degradable component partially or fully degrades.

In another and/or alternative aspect of the present invention, there is provided a degradable component that includes a plurality of tracer elements, and wherein the plurality of tracer elements is the same.

In another and/or alternative aspect of the present invention, there is provided a degradable component that includes a plurality of tracer elements, and wherein some of the tracer elements are different from some of the other tracer elements.

In another and/or alternative aspect of the present invention, there is provided a method for determining 1) whether a degradable component has begun to degrade, 2) the degree to which the degradable component has degraded, 3) whether the one or more particular regions of the degradable component has begun to degrade and/or the degree to which such one or more particular regions have degraded, and/or 4) whether the degradable component has been sufficiently removed from a location (e.g., a location in a well, etc.), wherein such method comprises the steps of a) providing a degradable component (e.g., a tool, a component of a tool, a valve, a plug, frac ball, etc.); b) providing one or more tracer elements (and if a plurality of tracer elements the tracer elements can be the same or different) that are incorporated on and/or in the degradable component, wherein the one or more tracer elements are configured to be released from the degradable component when the degradable component partially or fully degrades; c) exposing the degradable component to fluid (e.g., a flowing stream of fluid, etc.) which causes the degradable component to partially or fully degrade and thereby partially or fully releasing one or more tracers element from the degradable component that previously located on and/or in the degradable component; and d) providing a detection arrangement (e.g., sensor, testing lab, visual inspection, etc.) to detect the presence and/or concentration of one or more tracer elements prior to release from the degradable component and/or after released from the degradable component due to the partial or full degradation of the degradable component. The sensor can be located at a location away from the degradable component (e.g., one or more feet to one or more miles from the location of the degradable component [and all values and ranges of such distances therebetween]); however, this is not required. For example, if fluid (e.g., water, a salt solution, polymer solution, etc.) is flowed into a location of the degradable component (e.g., well, etc.), the flowing fluid can be used to cause the degradable component to partially or fully degrade, thereby causing one or more tracer elements to be released from the degradable component and to flow with the fluid downstream from the degradable component. At some location downstream of the degradable component, the fluid can be 1) analyzed by a sensor to detect the presence of one or more tracer elements; 2) samples of the fluid can be taken and tested by a sensor, some other testing device, or in a lab to test and/or detect the presence of one or more tracer elements; and/or 3) visually detected (e.g., a person seeing a color change in the fluid, etc.) to thereby detect the presence of one or more tracer elements. The detection of the presence of a tracer element and/or the amount of detected tracer element can be used to determine that 1) the degradable component has not degraded, 2) the degradable component has not sufficiently degraded, 3) a certain portion of the degradable component has not degraded, 4) a certain portion of the degradable component has begun to degrade, 5) the degree of degradation of one or more portions of the degradable component, and/or 6) the degradable component has sufficiently degraded. As can be appreciated, the method can be used to determine the degradation status of a plurality of degradable components that are the same or different. Different tracer elements can be used to differentiate the degree of degradation of different components and/or different regions of components.

In another and/or alternative aspect of the present invention, the detection arrangement can be located at or near degradable component (e.g., in contact with the degradable component to less than a foot from the degradable component [and all values and ranges of such distances therebetween]).

In another and/or alternative aspect of the present invention, the detection arrangement can be located at the surface of a well site and/or on the surface above the location of the degradable component that includes the one or more tracer elements.

In another and/or alternative aspect of the present invention, the detection arrangement can be located remotely from the degradable component (e.g., one or more feet to one or more miles from the location of the degradable component [and all values and ranges of such distances therebetween]).

In another and/or alternative aspect of the present invention, the composition of the fluid and/or the flowrate of the fluid to which the degradable component is exposed can be used to 1) control a rate of degradation of the degradable component, 2) measure or estimate dissolution rates of the degradable component, 3) measure or estimate the degree of degradation of the degradable component, and/or 4) measure or estimate flow rates of fluids through specific fluid or zones in a well due to the progression of dissolution or degradation of the degradable component. As such, tracer elements that are the same or different from the one or more tracer elements in the degradable component can be inserted into the fluid so as to 1) determine if the fluid has encountered one or more of the degradable components, 2) the flow rate of the fluid about the one or more of the degradable components, and/or the flow of fluid through one or more regions of a well.

In another and/or alternative aspect of the present invention, a detection arrangement is used to assure that a degradable component has properly degraded by discretely locating the tracer element on and/or in one or more locations of the degradable component, detecting the presence of the tracer element, and/or by estimating the total amount of the tracer element released from the degradable component.

In another and/or alternative aspect of the present invention, the degradable component can be formed of a plastic material or metal material (magnesium, magnesium alloy, aluminum alloy, etc.) which may or may not include a coating material and/or one or more additives. Non-limiting examples of degradable metal materials are disclosed in US Publication No. 2015/0239795 (Magnesium alloy that contains at least at least 30 wt % magnesium, typically greater than 50%, and more typically at least about 70%. The metals that can be included in the magnesium alloy can include, but are not limited to, aluminum, calcium, lithium, manganese, rare earth metal, silicon, SiC, yttrium, zirconium and/or zinc. Non-limiting examples of metals or metal alloys other than magnesium that are degradable metal alloys include aluminum alloys (e.g., aluminum alloys including 75+% aluminum and one or more of bismuth, copper, gallium, magnesium, indium, silicon, tin, and/or zinc); calcium; Ca-Mg, Ca-Al; and Ca-Zn.); US Publication No. 2015/0299838 (magnesium or magnesium alloy constitutes about 50.1 wt % 99.9 wt % of the magnesium composite and one or more additives such as copper, nickel, cobalt, titanium, iron, wherein the one or more additives generally have an average particle diameter size of at least about 0.1 microns, typically no more than about 500 microns and a higher melting point that magnesium.); and US Publication No. 2015/0240337 (A metal cast structure wherein the grain boundary composition and the size and/or shape of the insoluble phase additions can be used to control the dissolution rate of such composite. The composition of the grain boundary layer can optionally include two added insoluble particles having a different composition with different galvanic potentials, either more anodic or more cathodic as compared to the base metal or base metal alloy. The base metal or base metal alloy can include magnesium, zinc, titanium, aluminum, iron, or any combination or alloys thereof. The added insoluble particles that have a more anodic potential than the base metal or base metal alloy can optionally include beryllium, magnesium, aluminum, zinc, cadmium, iron, tin, copper, and any combinations and/or alloys thereof. The insoluble particles that have a more cathodic potential than the base metal or base metal alloy can optionally include iron, copper, titanium, zinc, tin, cadmium lead, nickel, carbon, boron carbide, and any combinations and/or alloys thereof. The grain boundary layer can optionally include an added component that is more cathodic as compared to the base metal or base metal alloy. The composition of the grain boundary layer can optionally include an added component that is more cathodic as compared to the major component of the grain boundary composition. The grain boundary composition can be magnesium, zinc, titanium, aluminum, iron, or any combination of any alloys thereof. The composition of the grain boundary layer can optionally include an added component that is more cathodic as compared to the major component of the grain boundary composition and the major component of the grain boundary composition can be more anodic than the grain composition. The cathodic components or anodic components can be compatible with the base metal or base metal alloy in that the cathodic components or anodic components can have solubility limits and/or do not form compounds. The component (anodic component or cathodic component) can optionally have a solubility in the base metal or base metal alloy of less than about 5% (e.g., 0.01-4.99% and all values and ranges therebetween), typically less than about 1%, and more typically less than about 0.5%. The composition of the cathodic components or anodic components in the grain boundary can be compatible with the major grain boundary material in that the cathodic components or anodic components have solubility limits and/or do not form compounds. The strength of metal cast structure can optionally be increased using deformation processing and a change dissolution rate of less than about 20% (e.g., 0.01-19.99% and all values and ranges therebetween), typically less than about 10%, and more typically less than about 5%. The ductility of the metal cast structure can optionally be increased using nanoparticle cathode additions. The metal cast structure can optionally include chopped fibers.); all of which are incorporated herein by reference. A non-limiting example of degradable plastic or polymer materials is disclosed in US 2016/0137912 (The expandable composite material can include one or more polymer materials selected from the group consisting of polyacetals, polysulfones, polyurea, epoxys, silanes, carbosilanes, silicone, polyarylate, and polyimide. The expandable material can include one or more materials selected from the group consisting of Ca, Li, CaO, Li₂O, Na₂O, Fe, Al, Si, Mg, K₂O and Zn. The expandable material generally ranges in size from about 106 μm to 10 mm. The expandable composite material can include one or more catalysts for accelerating the reaction of the expandable material; however, this is not required. The catalyst can include one or more materials selected from the group consisting of AlC1₃ and a galvanically active material. The expandable material can include strengthening and/or diluting fillers; however, this is not required. The strengthening and/or diluting fillers can include one or more materials selected from the group consisting of fumed silica, silica, glass fibers, carbon fibers, carbon nanotubes and other finely divided inorganic material. The expandable material can include a surface coating or protective layer that is formulated to control the timing and/or conditions under which the reaction or expanding occurs; however, this is not required. The surface coating can be formulated to dissolve when exposed to a controlled external stimulus (e.g., temperature and/or pH, chemicals, etc.). The surface coating can be used to control activation of the expanding of the core or core composite. The surface coating can include one or more materials such as, but not limited to, polyester, polyether, polyamine, polyamide, polyacetal, polyvinyl, polyureathane, epoxy, polysiloxane, polycarbosilane, polysilane, and polysulfone. The surface coating generally has a thickness of about 0.1 μm to 1 mm and any value or range therebetween.), which is incorporated herein by reference.

One non-limiting object of the present invention is the provision of a degradable component that includes one or more tracer elements.

Another non-limiting object of the present invention is the provision of a system and method of detecting or estimating whether a degradable component has properly degraded.

Another non-limiting object of the present invention is the provision of a degradable component for use in subterranean operations wherein the degradable component includes one or more tracer elements that are released upon the partial or full dissolution of the degradable component, and which the one or more tracer elements can be detected at the surface to determine the proper removal or degradation of the degradable component.

Another non-limiting object of the present invention is the provision of a degradable component wherein the one or more tracer elements are incorporated uniformly throughout the degradable component, added to specific locations in the degradable component, or placed at different depths within the degradable component.

Another non-limiting object of the present invention is the provision of a degradable component that includes a single tracer element or different tracer elements.

Another non-limiting object of the present invention is the provision of a degradable component wherein the tracer element is uniformly dispersed in the degradable component or is located in one or more regions of the degradable component or is concentrated in one or more regions of the degradable component.

Another non-limiting object of the present invention is the provision of a degradable component wherein the degradable component includes the addition of one or more tracer elements in an interior of the degradable component for the purpose of verifying and/or assuring that the degradable component has sufficiently degraded and/or dissolved.

Another non-limiting object of the present invention is the provision of a degradable component wherein the tracer element is less than a micron is size.

Another non-limiting object of the present invention is the provision of a degradable component wherein the type and/or amount of one or more tracer elements used in a particular degradable component is non-limiting.

Another non-limiting object of the present invention is the provision of a degradable component wherein the tracer elements can be 1) uniformly dispersed throughout a particular component, 2) concentrated in one or more regions of a particular component, and/or 3) include different types of tracer elements in different regions of a particular component.

Another non-limiting object of the present invention is the provision of a degradable component wherein the tracer element is incorporated in the degradable component and is designed to be released during or after the partial or full degradation of the degradable component.

Another non-limiting object of the present invention is the provision of a degradable component wherein one or more tracer elements are placed in an internal cavity of the degradable component and a degradable plug or cap is used to close the cavity; upon degradation of the cap or plug, the tracer elements in the cavity are partially or fully released from the cavity.

Another non-limiting object of the present invention is the provision of a degradable component wherein the tracer element is designed, after the degradable component partially or fully degrades, to release from the degradable component and be carried with fluid flow to a location at some distance from where such one or more tracer elements are released from the degradable component, and which tracer elements can be detected once such tracer elements are transported to a different location from the location of the degradable component.

Another non-limiting object of the present invention is the provision of a degradable component wherein different tracer elements are used in different regions or zones of a degradable component to provide information as to the degree to which a degradable component has degraded and/or whether a particular region of a degradable component has degraded and/or the degree to which it has been degraded.

Another non-limiting object of the present invention is the provision of a degradable component wherein different types of tracer element are incorporated and/or positioned at different regions of a degradable component.

Another non-limiting object of the present invention is the provision of a degradable component wherein different tracer elements are used in different degradable components.

Another non-limiting object of the present invention is the provision of a degradable component wherein the tracer element can be chosen from one or more microRFID, magnetic wires, nanowires, magnetic particles, fluorescing, and phosphorescent compounds and/or particles; and/or from compounds or molecules that can include stable isotopes, radioactive isotopes, rare earth or other specific elements, as well as compounds with high sensitivity in mass spectroscopy or other analytical technique that is sensitive to ppb levels.

Another non-limiting object of the present invention is the provision of a degradable component wherein the tracer element can be in the form of one or more nanomaterials and/or types of nanomaterials such as, but not limited to, nanotubes, nanocomposites, nanohorns, functionalized nanotubes, metalized nanotubes, combinations of different nanomaterials, and combinations of different functionalized nanotubes and/or metalized nanotubes.

Another non-limiting object of the present invention is the provision of a degradable component wherein the tracer element can include one or more RFID and/or other nano-device.

Another non-limiting object of the present invention is the provision of a degradable component wherein the tracer element includes one or tracer molecules, and/or one or more different tracer molecules.

Another non-limiting object of the present invention is the provision of a degradable component wherein the tracer element can be incorporated into degradable materials by encapsulation to create controlled release detection and/or allow placement and then release at different depths and within a well formation.

Another non-limiting object of the present invention is the provision of a system for assuring performance of a degradable component that includes a) a degradable material partially or fully forming the degradable component, and b) one or more tracer elements and/or types of tracer elements incorporated on and/or in the degradable component, wherein the one or more tracer elements are configured to be released from the degradable component when the degradable component partially or fully degrades.

Another non-limiting object of the present invention is the provision of a method of detecting one or more tracer elements and/or types of tracer elements at some distance from a location of a degradable component.

Another non-limiting object of the present invention is the provision of a degradable component wherein the tracer element is a stable isotope/element that is incorporated on and/or in a degradable component which is detectable using analytical techniques prior to, during, and/or after the degradable component partially or fully degrades.

Another non-limiting object of the present invention is the provision of a degradable component wherein the tracer element is an oxide or other type of compound, such as a rare earth oxide, that is incorporated on and/or in a degradable component which is detectable using analytical techniques prior to, during, and/or after the degradable component partially or fully degrades.

Another non-limiting object of the present invention is the provision of a degradable component wherein the tracer element is a rare earth material not normally found in the formation fluids that is incorporated on and/or in a degradable component which is detectable using analytical techniques prior to, during, and/or after the degradable component partially or fully degrades.

Another non-limiting object of the present invention is the provision of a degradable component wherein the tracer element is incorporated uniformly throughout the degradable component.

Another non-limiting object of the present invention is the provision of a degradable component wherein the tracer element is located on and/or within one or more specific area or regions of the degradable component.

Another non-limiting object of the present invention is the provision of a degradable component wherein the degradable component includes one or more tracer elements in an amount of at least about 0.01 grams.

Another non-limiting object of the present invention is the provision of a degradable component wherein the degradable component includes one or more tracer elements in an amount of 0.01-10 grams (and all values and ranges therebetween).

Another non-limiting object of the present invention is the provision of a degradable component wherein the tracer element is in the form of an RFID tag, magnetic wire, or other information carrying device that is incorporated on and/or in a degradable component which is detectable using analytical techniques prior to, during, and/or after the degradable component partially or fully degrades.

Another non-limiting object of the present invention is the provision of a degradable component wherein the tracer element is in the form of a tracer molecule or element that is incorporated on and/or in a degradable component which is detectable using analytical techniques prior to, during, and/or after the degradable component partially or fully degrades.

Another non-limiting object of the present invention is the provision of a degradable component wherein the degradable component includes a plurality of tracer elements, and wherein the plurality of tracer elements is the same.

Another non-limiting object of the present invention is the provision of a degradable component wherein the degradable component includes a plurality of tracer elements, and wherein some of the tracer elements are different from some of the other tracer elements.

Another non-limiting object of the present invention is the provision of a method for determining 1) whether a degradable component has begun to degrade, 2) the degree to which the degradable component has degraded, 3) whether the one or more particular regions of the degradable component has begun to degrade and/or the degree to which such one or more particular regions have degraded, and/or 4) whether the degradable component has been sufficiently removed from a location, wherein such method comprises the steps of a) providing a degradable component, b) providing one or more tracer elements (and if a plurality of tracer elements the tracer elements can be the same or different) that are incorporated on and/or in the degradable component, wherein the one or more tracer elements are configured to be released from the degradable component when the degradable component partially or fully degrades, c) exposing the degradable component to fluid which causes the degradable component to partially or fully degrade and thereby partially or fully releasing one or more tracers element from the degradable component that was previously located on and/or in the degradable component, and d) providing a detection arrangement (e.g., sensor, testing lab, visual inspection, etc.) to detect the presence and/or concentration of one or more tracer elements prior to release from the degradable component and/or after released from the degradable component due to the partial or full degradation of the degradable component.

Another non-limiting object of the present invention is the provision of a degradable component wherein 1) fluid can be analyzed by a sensor to detect the presence of one or more tracer elements, 2) samples of the fluid can be taken and tested by a sensor, some other testing device, or in a lab to test and/or detect the presence of one or more tracer elements, and/or 3) fluid can be visually detected to thereby detect the presence of one or more tracer elements.

Another non-limiting object of the present invention is the provision of a method of detection of the presence of a tracer element and/or the amount of detected tracer element to determine that 1) the degradable component has not degraded, 2) the degradable component has not sufficiently degraded, 3) a certain portion of the degradable component has not degraded, 4) a certain portion of the degradable component has begun to degrade, 5) the degree of degradation of one or more portions of the degradable component, and/or 6) the degradable component has sufficiently degraded.

Another non-limiting object of the present invention is the provision of a method used to determine the degradation status of a plurality of degradable components that are the same or different. Different tracer elements can be used to differentiate the degree of degradation of different components and/or different regions of components.

Another non-limiting object of the present invention is the provision of a method wherein the composition of the fluid and/or the flowrate of the fluid to which the degradable component is exposed can be used to 1) control a rate of degradation of the degradable component, 2) measure or estimate dissolution rates of the degradable component, 3) measure or estimate the degree of degradation of the degradable component, and/or 4) measure or estimate flow rates of fluids through specific fluids or zones in a well due to the progression of dissolution or degradation of the degradable component.

Another non-limiting object of the present invention is the provision of a method of using tracer elements that are the same or different from the one or more tracer elements in the degradable component are inserted into the fluid so as to 1) determine if the fluid has encountered one or more of the degradable component, 2) determine the flow rate of the fluid about the one or more of the degradable component, and/or the flow of fluid through one or more regions of a well.

Another non-limiting object of the present invention is the provision of a method to assure that a degradable component has properly degraded by discretely locating the tracer element on and/or in one or more locations of the degradable component, detecting the presence of the tracer element, and/or by estimating the total amount of the tracer element released from the degradable component.

Another non-limiting object of the present invention is the provision of a tracer element that can be added into a pocket or cavity that has been machined into a tool or degradable component in an amount such that when the tool or degradable component partially or fully degrades, the tracer elements generates a readily detectable signal or is present in a concentration in the flowback or produced water that can be readily detected.

Another non-limiting object of the present invention is the provision of a tracer element that can be in a tool or degradable component in an amount such that when the tool or degradable component partially or fully degrades, the tracer elements generates a readily detectable signal or is present in a concentration in the flowback or produced water that can be readily detected.

Another non-limiting object of the present invention is the provision of tracer elements, such as chemical tracer elements, molecular compound tracer elements, elemental tracer elements, or isotope tracer elements, are present in an amount in and/or on the tool or degradable component so as to be detectable above the detection thresholds in the flowback water during the initial flowback, and/or later during produced water (e.g., water that flows through the well). Flowback normally occurs as part of the process of putting the well into production, generally from one day and three weeks after completing the well. Chemical tracers normally are detectable at sub-PPM to PPB levels, using available detection technologies. Radioisotopes generally have lower detection thresholds than salts or molecular tracers. The target level of tracer elements, such as chemical tracer elements, molecular compound tracer elements, elemental tracer elements, or isotope tracer elements in the flowback or produced water is about 0.01-10 ppm in the expected volumetric flow of flowback water and/or later during produced water.

Another non-limiting object of the present invention is the provision of tracer elements, such as chemical tracer elements, molecular compound tracer elements, elemental tracer elements, or isotope tracer elements are present in an amount in and/or on the tool or degradable component of about 5-500 grams (and all values and ranges therebewteen) in a tool or degradable component, depending on expected water volume and flow duration during the dissolution and tracer release process.

Another non-limiting object of the present invention is the provision of tracer elements that is present in an amount in and/or on the tool or degradable component of at least about 0.01 wt. % of the tool or degradable component and less than 50 wt. % of the tool or degradable (and all values and ranges therebewteen). In one non-limiting embodiment, the tracer element in the form of a chemical tracer elements, molecular compound tracer elements, elemental tracer elements, and/or isotope tracer elements is generally present in an amount in and/or on the tool or degradable component of about 0.01 wt. % to 45 wt. % (and all values and ranges therebetween) of the tool or degradable component, and typically about 0.05-40 wt. % of the tool or degradable component.

Other objects, advantages, and novel features of the present invention will become apparent from the following detailed description of the invention when considered in conjunction with the accompanying drawings.

### BRIEF DESCRIPTION OF THE DRAWINGS

Reference may now be made to the drawings which illustrate various non-limiting embodiments that the invention may take in physical form and in certain parts and arrangement of parts wherein:

FIG. 1 illustrates a body formed of a degradable matrix that includes a plurality of tracer elements in the form of oxide particles, and wherein the body is represented in three states: 1) the degradable matrix of the body has not dissolved or degraded; 2) the degradable matrix of the body has begun to degrade, but no tracer element has been released from the body; and 3) the degradable matrix of the body has degraded to a point wherein tracer element has been released from the body.

FIG. 2 illustrates a body formed of a degradable material that includes a cavity filled with a plurality of tracer elements in the form of tracer tags, particles, and/or compound, and wherein a degradable plug is used to retain the tracer element in the cavity until the degradable material and/or the degradable plug has sufficiently degraded.

FIG. 3 illustrates a body formed of a degradable material wherein the tracer elements are uniformly dispersed throughout the degradable material.

FIG. 4 illustrates a body formed of a degradable material wherein the tracer elements are concentrated in a particular region of the degradable material, namely the center of the degradable material.

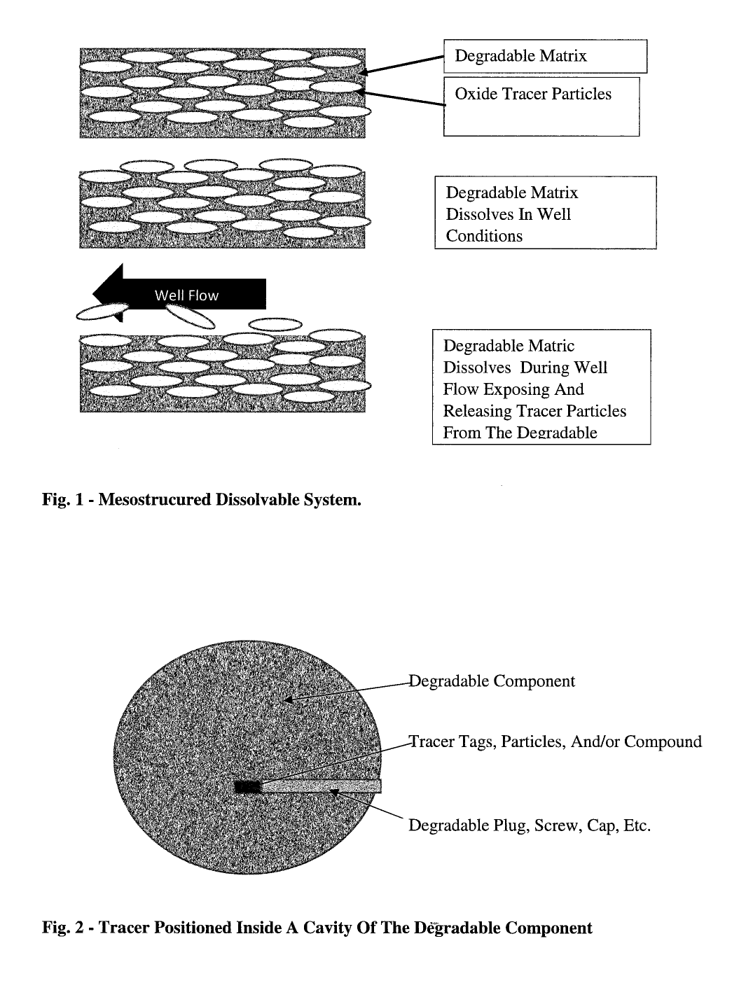

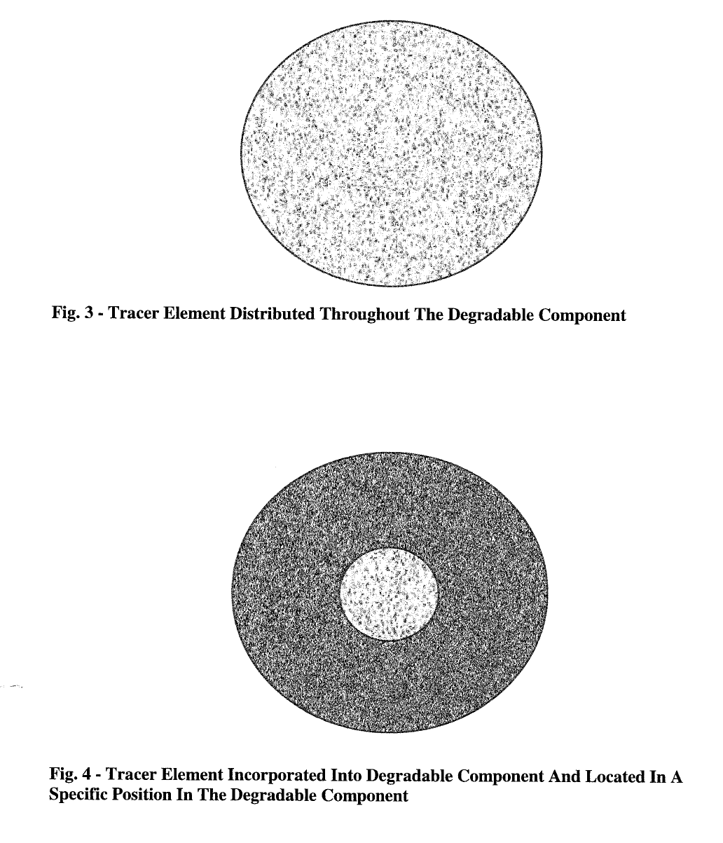

### DESCRIPTION OF NON-LIMITING EMBODIMENTS

The present invention relates to the enhanced use of degradable or dissolving tools and devices used in subterranean operations such as drilling, completion, and stimulation operations used in enhanced geothermal, oil and gas, and waste disposal (injection) operations wherein the degradable components include tracer elements that are released upon the partial or full dissolution of the degradable component, and which can be detected at the surface to ensure the desired degradation or removal of the degradable component.

In accordance with the present invention, chemical tracers can be added into a pocket or cavity that has been machined into a tool and/or the chemical tracer can be added as a constituent or additive to the degradable material formulation of the tool to generate a readily detectable signal or concentration in the flowback or produced water. Tracer chemicals or isotopes need to be detectable above the detection thresholds in the flowback water during the initial flowback, or later during produced water for a slower dissolving tool or tool design to release tracers during production. Flowback normally occurs as part of the process of putting the well into production, generally from one day and three weeks after completing the well. Chemical tracers normally are detectable at sub-PPM to PPB levels, using available detection technologies. Radioisotopes generally have lower detection thresholds than salts or molecular tracers. The design and size of the cavity in the tool (or concentration in the tool for rare earth or radioisotopes incorporated into the alloy) is selected to result in a target level of chemical tracer in the flowback or produced water of from about 0.01-10 ppm in the expected volumetric flow during the time of dissolution or flow being analyzed. To obtain such concentrations in the flowback or produced water, the concentration of the tracer chemical in the tool is generally about 5-500 grams in a tool, depending on expected water volume and flow duration during the dissolution and tracer release process. Generally, the degradable component contains at least 1 wt. % tracer chemical. In one non-limiting specific embodiment, the degradable component contains about 1-45 wt. % tracer chemical (and all values and ranges therebetween).

### EXAMPLE 1

### Oil/Water Soluble Mesostructured Degradable Tracer

A mesostrucured tracer/degradable system is provided wherein the degradable component is soluble in the well (aqueous) fluid, thereby exposing the tracer element to the well flow conditions. The tracer element includes stable isotopes not common in the well formation, and which the tracer element is normally in the form of oxide or intermetallic particles. The tracer element is released from the degradable component as the degradable component degrades. The degradable component can be formed of a polymeric and/or metallic material. The released tracer elements can be analyzed on-site by testing the fluid flow or back flow of fluid from the well or by sending a sample of the fluid containing the tracer elements to an outside lab, typically a lab that uses high resolution GC-MS techniques; however, this is not required.

For example, the tracer element can be formed from rare earth oxide nanoparticles (CeO, Ge₂O₃, Sm₂O₃, Nd₂O₃, etc.) that are readily prepared using sol-gel synthesis and incorporated into the degradable component (e.g., polymeric degradable components, metallic degradable components, etc.). By sampling the flow or flowback water during completion, or at the start of well production, and partially evaporating the water, sensitivities in the ppt range can be achieved to detect the tracer elements in the tested fluid. The concentration of the tracer elements in the fluid is directly related to the volume of degradable component that has degraded when knowing the total flowback and a loss correction factor.

In the case of soluble degradable components such as polymerics, the tracer elements (e.g., oxide tracer particles, etc.) are released in proportion to the flow rate of the well section, and as the tracer elements are released, more polymer of the degradable component will be exposed to be dissolved in the well. By adding tracer elements to the degradable component such that they constitute a large percentage of the surface of the degradable component on dissolution, flow sensitivity (e.g., flow rates, etc.) of the fluid in the well can be increased by the detection of the tracer elements.

The detection of the tracer element can provide instantaneous degradation rates (how fast the degradation of the degradable component) as well as cumulative degradation of the degradable component (either sampling from total flowback, or averaged over total flowback, such as a sample from each container of flowback fluid). By adding different tracer elements to different zones or locations of the degradable component, the degradation of each zone of the degradable component can be determined. In one non-limiting example, the degradable component is a frac ball having a diameter of 1-5 inches. The tracer elements can be uniformly dispersed throughout the frac ball or be localized in the frac ball (e.g. inserted into a cavity in the frac ball). The frac ball can be formed of a metal or plastic material. The tracer element constituted less than 30 wt. % of the frac ball.

### EXAMPLE 2

### Localized Tracer Incorporation

The system as set forth in Example 1 releases tracer elements continuously during degradation/dissolution of the degradable component, and requires knowledge of the total flow of fluid past the degradable component and a recovery of a known percentage of the tracer element to assess the complete or desired amount of removal of the degradable component in the well. Such information can be facilitated by adding the tracer element at a select depth in the degradable component (such as the center of a degradable component [e.g., frac ball, etc.]). In such an arrangement, the tracer element can be added to the degradable component in a more concentrated form; however, this is not required if the release is detectable at lower concentrations. The insertion of the tracer element can be incorporated in the interior of the degradable component by various processes, for example, by drilling a hole or machining a cavity into the degradable component, placing the tracer element in the bottom of the cavity, and then plugging the hole. In this manner, any detection of the tracer element confirms full or sufficient removal of the degradable component. An alternate approach is to place the tracer in a pocket between, or at the intersection of, two components; for example, below the element or seal and the mandrel in a dissolvable frac or bridge plug such that when the tracer element is detected, it is known that the degradable component has been sufficiently degraded. The tracer element constituted less than 25 wt. % of the frac ball, and typically less than 10 wt. %. For example, the tracer element can constitute about 1-25 g.

### EXAMPLE 3

A dissolvable metal frac ball having outer dimensions of 3.750 inches +/− 0.003 inches is machined to form a hollow core having dimensions of 0.75 inch×2.5 inch. The hollow core thus constitutes less than 15 vol. % of the frac ball. The upper 1 inch of the cavity is machined with female NPT threads to accept a plug. One or more microRFID tags (e.g., 1-5 microRFID tags) having dimensions to fit into the hollow core (e.g., 0.6″ dia×0.1″) are coated with a coating to protect the one or more microRFID tags and/or provide buoyancy to the one or more microRFID tags so that the microRFID tags can float in the flowing water after being released form the degradable component. The coating is typically a non-degradable coating in the well fluid. The coating thickness is generally at least about 0.005 inches and typically about 0.01 inches to 0.1 inches (and all values and ranges therebetween). One non-limiting coating is a polyurethane coating. The coating can optionally can include about 0.1-70% vol. % (and all values and ranges therebetween) additive (e.g., microballoons, hollow spheres, high buoyance materials, etc.) to increase the buoyancy of the coating. One non-limiting additive are glass microballoons. In one non-limiting example, the microRFID tag can be coated with 0.02-0.05 inches of polyurethane which optionally contains about 30-35 vol. % glass microballoons. A dissolvable metal plug with matching male NPT threads is threaded into the cavity to form a seal to seal the cavity, then surface machined to the frac ball spherical surface to meet the frac ball diameter specifications. The frac ball is used during a normal stimulation process, and allowed to dissolve over 1 to 10 day period (e.g., 3-5 day period). A screening device is placed in the discharge of the flowback pipe, or a solids catcher is used in the flowback line to collect solids, typically greater than ⅛ inch or ¼ inch. The screening device is selected and designed so as to capture the microRFID tags. The screen, filter, or solids catcher are checked periodicially or at the end of flowback as the well is close to connect to the production equipment. The information on the RFID tags is read by a portable reader to positively confirm complete dissolution of the frac ball, and to collect any other information the RFID tag or microcircuit has been constructed to collect. The tracer element constituted less than 25 wt. % of the frac ball, and typically less than 10 wt. %.

### EXAMPLE 4

A dissolvable metal frac ball having outer dimensions of 3.750 inches +/− 0.003 inches is machined to form a hollow core having dimensions of 0.75 inch×2.5 inch. The upper 1 inch of the cavity is machined with female NPT threads to accept a plug. 10-20 grams of a chemical tracer, such as, but not limited to, FFI 2300 from Spectrum Tracer Services, is then placed into the cavity. A dissolvable metal plug with matching male NPT threads is threaded into the cavity to form a seal, then surface machined to the ball spherical surface to meet the frac ball diameter specifications. The frac ball is used during a normal stimulation process, and allowed to dissolve over a 3-5 day period. Samples of the flowback water are collected periodically during completion, and sent to an analysis lab (in this case, Spectrum Tracer Services, LLC) for identification. Different tracers can be loaded into a series of frac balls (Spectrum Tracer Services, LLC has 41 FFI tracers available) to confirm that each stage of the well has completed dissolution of the frac balls in a particular section of the well. Identification of tracer elements from the toe stages confirmed that the well was open and flowing from all stages. The tracer element constituted less than 25 wt. % of the frac ball, and typically less than 10 wt. %.

In addition to chemicals detectable by analytical techniques (e.g., stable isotopes, high sensitivity molecules), microtags detectible using RF or other electromagnetic techniques can alternatively or additionally be used as the tracer element. One non-limiting example is to use a set of micro-RFID tags placed in the degradable component. The micto-RFID tags can be the only tracer elements or be used with other types of tracer elements (e.g. chemical tracers, etc.). A sufficient number of tags should be placed in the degradable component to ensure highly reliable detection in the flowback water or out-flowing water. These tracer elements can be detected in real time by flowing the produced/flowback fluid over or through a detection device. MicroRFID tags in the 100-300 micron range can be used and can be detected in a fluid flow using current detection technology. Medium or high frequency tags can be used, generally requiring recovery (such as by catching in a screen) during flowback and analysis, or low frequency tags, which are larger, have greater distance response particularly in water, and can more easily be analyzed on-line through the use of an antenna covering all or a portion of the flowback stream, with or without recovery of the tag. The tag can be engineered used to collect additional information, such as temperatures, salinity, pH, or other conditions occurring during the dissolution and exposure, and report those to the surface.

By adding unique tracer elements to different degradable components, the degradation and/or degradation rate of different degradable components can be independently monitored in the same flowback water. Also, by adding unique tracer elements to different regions of a degradable component, the degradation and/or degradation rate of a particular region or zone of degradable component can be independently monitored in the same flowback water.

The above described invention is most commonly used to assure removal of components such as frac balls, bridge plugs, perforators, sleeves, liners, pintles, seals, etc. The method and system of the present invention also or alternatively can be used to detect and identify flows from the formation, such as by flowing produced fluid through a device including a degradable component, after which detection of the tracer element can provide information on water flows and rates (by concentration versus total flow). The adding of different types and/or compositions of tracer elements to different degradable components that are located in different zones of a well allows total water flow to be identified from each zone in the well. Such information can be used to control production and intervention activities in the well.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall there between. The invention has been described with reference to the preferred embodiments. These and other modifications of the preferred embodiments as well as other embodiments of the invention will be obvious from the disclosure herein, whereby the foregoing descriptive matter is to be interpreted merely as illustrative of the invention and not as a limitation. It is intended to include all such modifications and alterations insofar as they come within the scope of the appended claims.

What is claimed:

1. A method of monitoring or confirming dissolution of a degradable component and open bore access in a well using a degradable or dissolvable tool component for use in subterranean operations that includes the steps of:
a. Providing a tool component that is partially or fully formed of a degradable material and includes one or more tracer elements;
b. Placing said tool component downhole into the bore or near-bore area of a well formation;
c. Causing said tool component to at least partially degrade or dissolve and to partially or fully release said one or more tracer elements from said tool component; and,
d. Recovering, collecting, monitoring or analyzing said one or more tracer elements to confirm dissolution or degradation of said tool component or a degree of dissolution or degradation of said tool component to thereby determine whether desired bore access has been obtained in said subterranean operation.

2. The method as defined in claim 1, wherein said degradable component is formed of a metallic or polymer material, said one or more tracer elements having a composition that is electromagnetically or chemically different from said degradable component, said one or more tracer elements located on a surface of said degradable component, incorporated in one or more regions of said degradable component, located in a cavity of said degradable component, or combinations thereof, said tracer element configured to be released from said degradable component when said degradable component partially or fully degrades.

3. The method as defined in claim 1, wherein said tool is a valve, plug, frac ball, flow diverter, pipe section or lining, block, or rod.

4. The method as defined in claim 1, wherein said degradable component includes a plurality of tracer elements, at least two of said tracer elements a) are different types of tracer elements, b) have a different composition, or a combination of a) and b).

5. The method as defined in claim 1, wherein said tracer element is uniformly distributed in said degradable component.

6. The method as defined in claim 1, wherein said tracer element is not uniformly distributed in said degradable component.

7. The method as defined in claim 1, wherein one or more of said tracer elements includes one or more components selected from the group consisting of a) piezoelectric material, b) identifier tag, c) dye, d) compound with high sensitivity in mass spectroscopy, e) element with high sensitivity in mass spectroscopy, f) carbon-based nanomaterials, g) tracer molecule, and h) water soluble salt.

8. The method as defined in claim 1, wherein said tracer element includes one or more tracer molecules selected from the group consisting of fluorescent molecule, UV-active molecule, isotopically-enriched molecule, radiolabeled molecule, radioactive molecule, metal particle, hydrophobic molecule, hydrophilic molecule, rare earth particle, phosphorescent particle, phosphorescent compound, fluorescent particle, fluorescent compound, stable isotope, ionizable molecule, fluorinated compound, sulfonated compound, and triheptylamine.

9. The method as defined in claim 1, wherein said tracer element includes one or more identifier tags selected from the group consisting of RFID, micro-resonant device, numerically-imprinted or other physical ID tag, and nano-device.

10. The method as defined in claim 9, wherein at least one of said identifier tags includes a buoyancy material to improve a buoyancy of said identified tag when in a liquid, said buoyancy material including a coating, an additive, or combination thereof.

11. The method as defined in claim 10, wherein said buoyancy material includes a coating on said identifier tag, said coating including a buoyant material or one or more hollow cavities that are used to reduce a density of said coating to near or below a wellbore fluid density.

12. The method as defined in claim 1, wherein said tracer element has a size of less than 1 micron.

13. The method as defined in claim 1, wherein said degradable component includes a cavity that includes one or more of said tracer elements, said cavity being covered or sealed with a plug on an opening of said cavity to prevent said tracer elements from escaping said cavity until said degradable component is partially or fully degraded.

14. The method as defined in claim 1, wherein said tool includes at least about 1 gram of said tracer element.

15. The method as defined in claim 1, wherein said tool includes up to about 25 grams of said tracer element.

16. The method as defined in claim 1, wherein multiple tool components are inserted into the well, a plurality of said tool components including one or more different tracer elements, and further including the step of confirming dissolution or degradation of tool components or a degree of dissolution or degradation of said tool component separately from one another and whether desired bore access has been obtained in a location of a particular said tool components.

17. A method for detecting the status of a fully or partially removable tool used in subterranean operations comprising the steps of:
a. providing a degradable or dissolvable tool that comprises a degradable base component that forms all or part of said tool and one or more tracer elements, said dissolvable base component formed of a metallic or polymer material, said one or more tracer elements having a composition that is different from said degradable base component, said one or more tracer elements located on a surface of said dissolvable base component, incorporated in one or more regions of said dissolvable base component, located in a cavity of said tool, or combinations thereof, said one or more tracer elements configured to be released from said dissolvable base component when said degradable base component partially or fully degrades;
b. positioning said tool in a subterranean environment;
c. providing a detection arrangement to detect a presence of one or more tracer elements, a concentration of one or more tracer elements, or combinations thereof;
d. causing said degradable base component to partially or fully degrade to enable one or more of said tracer elements to release from said tool; and,
e. detecting a presence, a concentration, or combinations thereof of said one or more tracer elements by said detection arrangement and using such information to determine i) an amount of degradation of said tool, ii) a desired amount of degradation of said tool, iii) whether desired bore access has been obtained in said subterranean operation, or some combination of i), ii) and iii).

18. The method as defined in claim 17, wherein said detection arrangement includes one or more arrangements selected from the group consisting of a sensor, a testing lab, and visual inspection.

19. The method as defined in claim 17, wherein said tracer element is a stable isotope/element which is detectable using analytical techniques.

20. The method as defined in claim 17, wherein said tracer element is a rare earth oxide.

21. The method as defined in claim 17, wherein said tracer element has an average particle size of less than 1 micron.

22. The method as defined in claim 17, wherein said tracer element is a RFID tag, magnetic wire, or other information carrying device.

23. The method as defined in claim 17, wherein said tracer element is a tracer molecule or element.
